(19) United States
(12) Patent Application Publication
(10) Pub. No.: US 2020/0275843 A1
(43) Pub. Date: Sep. 3, 2020

(54) INTEGRATED MEASUREMENT SYSTEM FOR MEASURING HEALTH-RELATED FITNESS FACTOR

(71) Applicants: Yong Seok JEE, Seosan-si (KR); Hanseo University Academic Cooperation Foundation, Seosan-si (KR)

(72) Inventor: Yong Seok JEE, Seosan-si (KR)

(73) Assignees: Hanseo University Academic Cooperation Foundation, Seosan-si (KR); Yong Seok JEE, Seosan-si (KR)

(21) Appl. No.: 16/782,765

(22) Filed: Feb. 5, 2020

(30) Foreign Application Priority Data
Feb. 28, 2019 (KR) 10-2019-0023645

(51) Int. Cl. A61B 5/024; A61B 5/00; G16H 10/60; G16H 80/00

(52) U.S. Cl. CPC A61B 5/024; A61B 5/4872; A61B 5/4519; A47C 1/024; G16H 10/60; G16H 80/00; A61B 5/0004

(57) ABSTRACT

Disclosed is an integrated measurement system for measuring health-related fitness factor and, more particularly, to an integrated measurement system for measuring health-related fitness factor, the integrated system being achieved by integrating separate health-related fitness measurement devices to be able to measure health-related fitness factors through one system, and raising convenience in management and guide by a manager and measurement by a measurer, and objectivity in a result by outputting measurement process and result in real time through a display.

### BACKGROUND OF THE INVENTION

### Field of the Invention

The present invention relates to an integrated measurement system for measuring health-related fitness factor and, more particularly, to an integrated measurement system for measuring health-related fitness factor, the integrated system being achieved by integrating separate health-related fitness measurement devices to be able to measure health-related fitness factors through one system, and raising convenience in management and guide by a manager and measurement by a measurer, and objectivity in a result by outputting measurement process and result in real time through a display.

### Description of the Related Art

Accurate measurements and tests must be performed to adequately improve an individual's health and physical strength.

For a precise physical examination, it should measure stamina factors such as a physical strength factor (grip strength) of the human body to express the exercise including the correct posture, a cardiovascular ability (cardiopulmonary endurance) to continuously maintain the exercise, and a neuromuscular function (flexibility) to adjust the exercise.

The physical strength can be divided into a health-related fitness and a skill related fitness.

The main goal of the health-related fitness is to improve the quality and quantity of life.

The improving of the physical strength through physical activity is very important for everyone, regardless of age or sex.

However, among the physical factors, the health-related fitness associated with the muscles is more important than the skill related fitness for middle-aged or more adults.

The health-related fitness is classified as five elements of muscular strength, endurance, flexibility, cardiopulmonary endurance and body composition (body composition test).

In general, the physical strength factor, which is focused on patients with middle-aged or adult diseases, is mainly the health-related fitness.

The health-related fitness is a fundamental factor in determining the quality of human life by providing the healthy spirit, that can actually maintain a strong body and overcome stress in leading life, and the ability capable of having a good working life.

Good health-related fitness does not mean that people do not get sick. However, it has been reported that the risk of occurrence of related diseases can be minimized if the health-related fitness is scientifically managed.

Thus, the present invention is to measure the above-mentioned health-related fitness and provide objective measurement results. That is, the present invention is provided to an integrated measuring device capable of performing various measurement items in one device, not a conventional measuring device for measuring only one health-related fitness factor, improving the measuring ease of the measuring device, and maximizing the managing ease of the manager for maintaining and managing the measuring device and the inspection institution.

That is, since the conventional measuring device is configured to measure only one health-related fitness factor, the measurer has to move between various places and locations in order to measure health-related fitness factors of various items.

In addition, since the measurer does not know in real time the measurement process and the results or the peoples do not know the average standard of the health-related fitness according to their age group, there are problems in that it is not motivated to improve the health-related fitness and only ends with a simple measurement.

Therefore, it needs a measuring device capable of maintaining and promoting health, providing a motivation, and measuring a variety of health-related fitness factors by easily managing the measuring device by means of the manager and the inspection institution and visually checking goodness or seriousness on the comprehensive health-related fitness in real time by means of the measurer.

Thus, as a prior art on the integrated measurement system for measuring health-related fitness factor, “a dynamic maximum strength measuring device and method thereof” of Korean Patent registration No. 10-0788968 (hereinafter referred to as “Patent Literature 1”) is disclosed.

Patent Literature 1 includes a load cell for measuring the strength of the applied force, a locking device for applying the force applied by the user to the load cell, a conversion formula for estimating a maximum repetition maximum (1-RM) from a maximum static muscular strength for each gender and age, and an indicator for inputting gender and age of the user from the users and estimating 1-RM of the corresponding user from the force used by the user in a fixed state of the weight measured through the load cell.

Thus, it can measure and evaluate the static maximum strength of the main joints and muscles of the human body by using each weight training machine. It relates to a method and a device of changing the static maximum strength to the dynamic maximum strength to be outputted, and a removable locking device used in the measuring device.

As another prior art, “an integrated measuring device for a student health fitness evaluation system” of Korean Patent registration No. 10-1033198 (hereinafter referred to as “Patent Literature 2”) is disclosed.

The Patent Literature 2 relates to an integrated measuring device for a student health fitness evaluation system capable of measuring various body information at the same time including a screen having a calibration figure thereon, two cameras vertically arranged to the screen, a balance meter for measuring the weight of a user and the distribution of the weight and having at least two load cells arranged symmetrically, and a central control unit for stitching the received image when there are several cameras, and analyzes the stitched image by referring to the horizontal and vertical reference lines displayed on the screen to determine the height and posture of the user. The central control unit calculates a center of pressure of the user from the distribution of the weight outputted from the balance meter and determines the posture of the user from the center of pressure, so that it can more easily and accurately measure various body information including the posture information of the user.

As described above, Patent Literature 1 includes the load cell for measuring the strength of the force and the indicator for estimating the dynamic maximum strength from the static maximum strength.

Patent Literature 1 relates to the method and the device of measuring and evaluating the static maximum strength of the main joints and muscles of the human body, and converting the evaluated static maximum strength into the dynamic maximum strength to be outputted.

Patent Literature 2 includes the balance meter having at least one camera and load cell.

Patent Literature 2 relates to the integrated measuring device capable of accurately measuring height, weight, body composition, grasping power, body balance and posture of the user at once.

That is, the Patent Literature 1 and Patent Literature 2 are commonly similar with the present invention in terms of the device for measuring the strength of the user. However, there are differences in the task (object of invention) to be solved and the solution means (components) for solving it, and the effects exerted by it.

Thus, the technical features of the present invention and the Patent Literature 1 and Patent Literate 2 are different from each other.

### PATENT LITERATURE

Patent Literature 1: Korean Patent Registration No. 10-0788968 (Dec. 18, 2007)

Patent Literature 2: Korean Patent Registration No. 10-1033198 (Apr. 28, 2011)

### SUMMARY OF THE INVENTION

The present invention has been made in an effort to solve the problems in the related art described above, and an object of the present invention is to provide an integrated measurement system that can easily measure various health-related fitness factors as one measurement system at the same position without specifically moving a measurer and a measurement device.

Another object of the present invention is to provide an integrated measurement system that enables a measurer to monitor in real time measurement process and result of his/her health-related fitness factors while measuring the health-related fitness factors.

Another object of the present invention is to provide an integrated measurement system that can provide a healthful mental stimulus so that a measurer can be given and maintain a motive about health management as the system enables the measurer to monitor his/her health-related fitness status in real time when measuring the health-related fitness factors.

Another object of the present invention is to provide an integrated measurement system that can be easily managed and measured by a manager and a measurer by securing convenience in management and measurement of a measurement device and objectivity in measurement process and result.

According to an aspect of the invention to achieve the object described above, there is provided an integrated measurement system for measuring health-related fitness factors, the integrated measurement system including:

a user seat enabling a measurer to sit and measure health-related fitness factors;

a health-related fitness measurement device measuring health-related fitness factors of the measurer sitting in the user seat;

a measurement information outputter outputting in real time measurement process and result of health-related fitness factors of the measurer that are measured by the health-related fitness measurement device so that the measurer and a manager can monitor; and

a measurement controller controlling the user seat, the health-related fitness measurement device, and the measurement information outputter,

wherein the health-related fitness measurement device includes:

a body configuration measurement unit that quantitatively measure body composition for each of a muscle amount and a body fat amount of the measurer;

a flexibility measurement unit that measures the degrees of maintenance and increase of the available ranges of joints and muscles of the measurer;

a cardiopulmonary endurance measurement unit that measures the ability to maintain the function of the circulatory system of the heart and the respiratory system of the lung of the measurer;

a muscular strength measurement unit that measures the strength of the muscles of the measurer and durability of the strength; and

a muscular endurance measurement unit that measures durability of predetermined muscular strength of a measurer for a long period of time, and

several health-related fitness factors of the measurer are easily measured through one system without moving the measurer and replacing measurement devices.

At this time, the integrated measurement system further includes a measurement result communication unit that shares and transmits a measurement date, personal information, and a measurement result of the measurer to a database of a measurement organization, a database of a public institution, a dedicated terminal of a manager, and a personal terminal of the measurer, whereby the measurement process and result of the health-related fitness factors of the measurer are induced to be professionally managed and indexes for healthcare are constructed on the basis of the collected measurement data.

In the meantime, it should be understood that the terminology or the words used in claims should not be interpreted in normally or lexically sense. It should be interpreted as meaning and concept consistent with the technical idea of the present invention, based on the principle that the inventor can properly define the concept of the term in order to describe its invention in the best way.

Therefore, the embodiments described in the present specification and the configurations shown in the drawings are only the most preferred embodiments of the present invention, and not all the technical ideas of the present invention are described. Therefore, it is to be understood that various equivalents and modifications are possible.

### BRIEF DESCRIPTION OF THE DRAWINGS

The above and other objects, features and advantages of the present invention will be more apparent from the following detailed description taken in conjunction with the accompanying drawings, in which:

FIG. 1 illustrates a concept diagram of an integrated measurement system for measuring health-related fitness factor according to the present invention;

FIG. 2 illustrates a block diagram of an integrated measurement system for measuring health-related fitness factor according to the present invention;

FIG. 3 illustrates an example of an integrated measurement system for measuring health-related fitness factor according to the present invention;

FIG. 4 is a flowchart simply showing the procedure of measuring health-related fitness factors using the integrated measurement system for measuring health-related fitness factors of the present invention;

FIG. 5 illustrates an example of outputting an information on a cardiopulmonary endurance of a measurer measured by a cardiopulmonary endurance measurement unit of an integrated measurement system for measuring health-related fitness factor of the present invention through a measurement information outputter; and

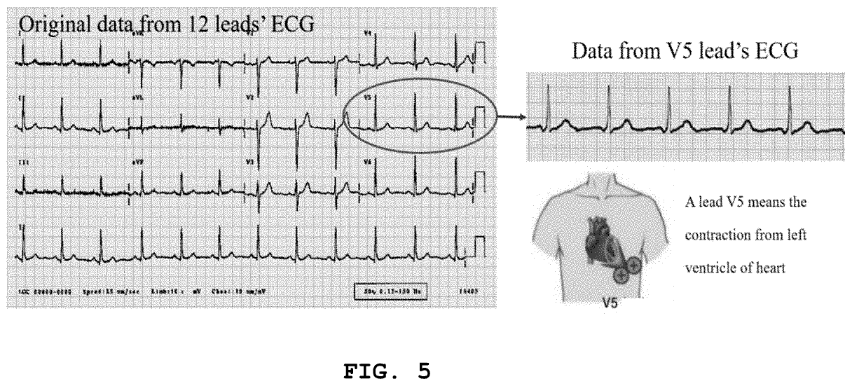

FIG. 6 illustrates an example of a method of measuring a cardiopulmonary endurance of a measurer by using a cardiopulmonary endurance measurement unit of an integrated measurement system for measuring health-related fitness factor of the present invention.

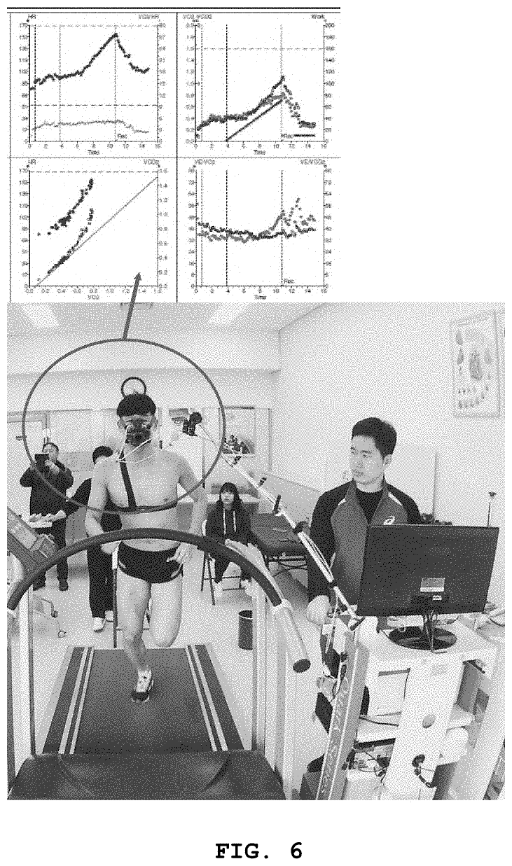

### REFERENCE SIGNS LIST

1: integrated measurement system for measuring health-related fitness factor
100: user seat
110: seat part
120: back
130: actuator
131: seat movement module
132: back angle adjustment module
200: health-related fitness measurement device
210: body configuration measurement unit
211: body configuration measurement module
212: body configuration measurement data transmitter
220: flexibility measurement unit
221: flexibility measurement module
222: flexibility measurement data transmitter
230: cardiopulmonary endurance measurement unit
231: cardiopulmonary endurance measurement module
232: cardiopulmonary endurance measurement data transmitter
240: muscular strength measurement unit
241: muscular strength measurement module
242: muscular strength measurement data transmitter
250: muscular endurance measurement unit
251: muscular endurance measurement module
252: muscular endurance measurement data transmitter
300: measurement information outputter
310: health-related fitness measurement information storage
311: body configuration measurement information storage module
312: flexibility measurement information storage module
313: cardiopulmonary endurance measurement information storage module
314: muscular strength measurement information storage module
315: muscular endurance measurement information storage module
320: health-related fitness information display
330: measurement method guide
331: body configuration measurement method guide module
332: flexibility measurement method guide module
333: cardiopulmonary endurance measurement method guide module
334: muscular strength measurement method guide module
335: muscular endurance measurement method guide module
400: measurement controller
B: measurement order provider
N: measurement result communication unit
N1: wire communication unit
N2: wireless communication unit
S1: dedicated terminal of a manager
S2: personal terminal of the measurer
DB1: database of measurement organization
DB2: database of public institution
TD: anti-fraud unit
TD1: measurer authentication module
TD2: measurer measurement process-temporal storage module
TD3: measurement-temporal stop module
TD4: measurement-continuing module
TD5: measurer measurement result connection module

### DETAILED DESCRIPTION OF THE PREFERRED EMBODIMENT

Hereafter, the function, configuration, and operation of an integrated measurement system for measuring health-related fitness factors according to the present invention are described in detail with reference to the accompanying drawings.

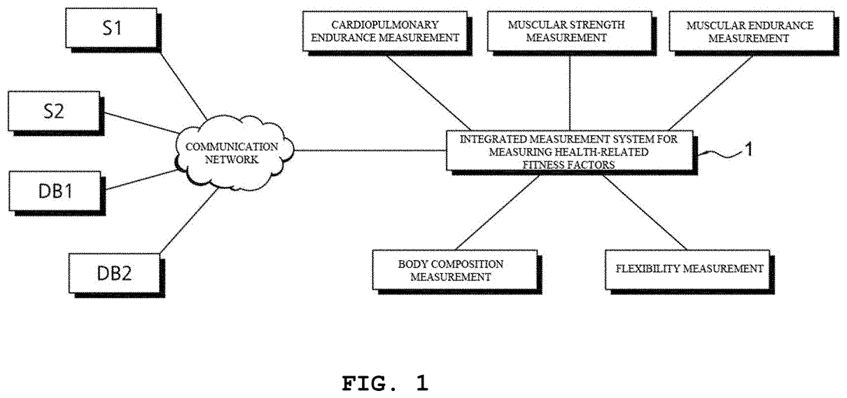

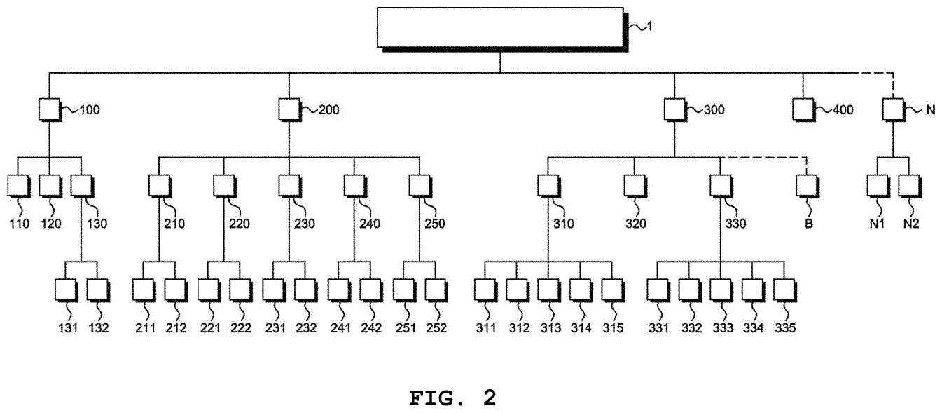

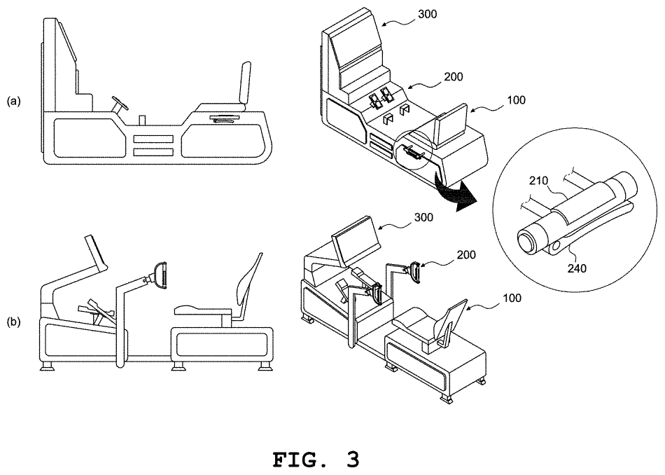

FIG. 1 illustrates a concept diagram of an integrated measurement system for measuring health-related fitness factor according to the present invention, FIG. 2 illustrates a block diagram of an integrated measurement system for measuring health-related fitness factor according to the present invention, and FIG. 3 illustrates an example of an integrated measurement system for measuring health-related fitness factor according to the present invention.

As shown in FIGS. 1 to 3, an integrated measurement system for measuring health-related fitness factors of the present invention includes:

a user seat (100) enabling a measurer to sit and measure health-related fitness factors;

a health-related fitness measurement device (200) measuring health-related fitness factors of a measurer sitting in the user seat (100);

a measurement information outputter (300) outputting in real time measurement process and result of health-related fitness factors of a measurer that are measured by the health-related fitness measurement device (200) such that a measurer and a manager can monitor; and

a measurement controller (400) controlling the user seat (100), the health-related fitness measurement device (200), and the measurement information outputter (300).

The health-related fitness measurement device (200) includes:

a body configuration measurement unit (210) that quantitatively measures body composition for each of a muscle amount and a body fat amount of a measurer;

a flexibility measurement unit (220) that measures the degrees of maintenance and increase of the available ranges of joints and muscles of a measurer;

a cardiopulmonary endurance measurement unit (230) that measures the ability to maintain the function of the circulatory system of the heart and the respiratory system of the lung of a measurer;

a muscular strength measurement unit (240) that measures the strength of the muscles of a measurer and durability of the strength; and

a muscular endurance measurement unit (250) that measures durability of predetermined muscular strength of a measurer for a long period of time.

Accordingly, it is possible to easily measure several health-related fitness factors of the measurer through one system without moving the measurer and replacing measurement devices.

That is, the integrated system of the present invention raises accuracy and convenience of measurement by enabling a measurer to sit in the user seat (100) and to measure and check several health-related fitness factors through one system using the health-related fitness measurement device (200) in accordance with information about the measurement items, the measurement order, and the measurement method of the health-related fitness factors that are output from the measurement information outputter (300).

In more detail, the user seat (100) in which a measurer sits to measure health-related fitness factors includes:

a seat part (110) in which a measurer can sit;

a back (120) that assists a measurer to maintain a sitting position by supporting the measurer's waist; and

an actuator (130) that moves the seat part (110) and the back (120) to optimal positions in accordance with the body shape and measurement items of a measurer.

Accordingly, a measurer can measure health-related fitness factors while maintaining an optimal position for measuring the health-related fitness factors.

The actuator (130) includes:

a seat movement module (131) that moves the seat part (110) forward, backward, and horizontally in accordance with the body shape and measurement items of a measurer; and

a back angle adjustment module (132) that adjusts the angle of the back (120) by rotating forward and backward in accordance with the body shape and measurement items of a measurer.

Accordingly, the measurer is assisted to maintain the optimal position for measuring health-related fitness factors.

On the other hand, the health-related fitness measurement device (200) that can measure health-related fitness factors that is, a body configuration (body composition examination), flexibility, cardiopulmonary endurance, muscular strength, and muscular endurance of a measurer includes:

the body configuration measurement unit (210) that quantitatively measure body composition for each of a muscle amount and a body fat amount of a measurer;

the flexibility measurement unit (220) that measures flexibility of joints and muscles of a measurer;

the cardiopulmonary endurance measurement unit (230) that measures the ability to maintain the function of the circulatory system of the heart and the respiratory system of the lung of a measurer;

the muscular strength measurement unit (240) that measures strength of the muscles of a measurer; and

the muscular endurance measurement unit (250) that measures muscular endurance of a measurer.

Accordingly, it is possible to acquire general information about the health-related fitness of the measurer.

The body configuration measurement unit (210) includes: a body configuration measurement module (211) that measures the body composition of a measurer; and a body configuration measurement data transmitter (212) that transmits in real time information about the body composition of a measurer measured by the body configuration measurement module (211) to the measurement information outputter (300).

The flexibility measurement unit (220) includes: a flexibility measurement module (221) that measures flexibility of a measurer; and a flexibility measurement data transmitter (222) that transmits in real time information about the flexibility of a measurer measured by the flexibility measurement module (221) to the measurement information outputter (300).

The cardiopulmonary endurance measurement unit (230) includes: a cardiopulmonary endurance measurement module (231) that measures cardiopulmonary endurance of a measurer; and a cardiopulmonary endurance measurement data transmitter (232) that transmits in real time information about the cardiopulmonary endurance of a measurer measured by the cardiopulmonary endurance measurement module (231) to the measurement information outputter (300).

The muscular strength measurement unit (240) includes: a muscular strength measurement module (241) that measures muscular strength of a measurer; and a muscular strength measurement data transmitter (242) that transmits in real time information about the muscular strength of a measurer measured by the muscular strength measurement module (241) to the measurement information outputter (300).

The muscular endurance measurement unit (250) includes: a muscular endurance measurement module (251) that measures muscular endurance of a measurer; and a muscular endurance measurement data transmitter (252) that transmits in real time information about the muscular endurance of a measurer measured by the muscular endurance measurement module (251) to the measurement information outputter (300).

The five health-related fitness factors described above are measured through the health-related fitness measurement device (200), and measurement information that is measured in real time is collected and stored in the measurement information outputter (300).

Briefly, the body configuration measurement unit (210) calculates a lean mass and a body fat mass of a human body using a body mass index, a waist-to hip ratio, a skin-fold method, Bone Scan and CT, and a somewhat simple examination method using bio-electricity impedance (in the present invention, as shown in FIG. 3, an integrated measurement system that employs the examination method using bio-electricity impedance is shown as an embodiment).

Further, the flexibility measurement unit (220) measures flexibility of a measurer using a distance technique that measures flexibility as a distance, an angle technique that measures flexibility as an angle, and an index technique that measures flexibility as an index (sit & reach of the distance technique is employed as an embodiment in the present invention).

The flexibility measurement method measures available ranges of joints and muscles with both talus bones of a measurer sitting in the seat part (110) closed to each other, two legs being open about 5 cm, feet attached to the integrated measurement system 1 for measuring health-related fitness factors, and the ends of hands to be positioned as low as possible by bending over while exhaling after taking a deep breath.

Further, the cardiopulmonary endurance measurement unit (230) performs measurement using a Grade Exercise Test (GXT) that uses a stepper method, and maximum oxygen uptake is used and measured as an evaluation element.

This is the most preferred method to evaluate the cardiovascular function, and the maximum oxygen uptake, which is an evaluation element, is determined with oxygen uptake per weight.

The maximum oxygen uptake of the measurer is derived by wirelessly measuring the number of heartbeats and a change in electrocardiogram of V5 after putting a polar-type belt on the chest of the measurer, and a change in inspiration of the measurer is derived by measuring an oxygen consumption amount (VO2) and a carbon dioxide discharge amount (VCO2) by wearing a mask on the mouth and nose.

The muscular strength measurement unit (240) measures static strength of a wrist muscle by the flexor digitorum and the flexor carpi of the measurer using a Smedley-type dynamometer through a grip strength test corresponding to static strength for measuring isometric strength of the measurer.

The muscular endurance measurement unit (250) measures muscular endurance of the measurer using sit-up, in which the muscular endurance is measured after adjusting the angle of the back (120) using the back angle adjustment module (132) with respect to the seat part (110) and producing an environment of the system in which the measurer can do sit-up.

On the other hand, the measurement information outputter (300) that outputs in real time measurement process and result of the health-related fitness factors of a measurer so that the measurer and a manager can check them includes: a health-related fitness measurement information storage (310) that stores in real time measurement process and result of the health-related fitness factors of a measurer that are sequentially measured through the health-related fitness measurement device (200); a health-related fitness information display (320) that outputs in real time the measurement process and result of the health-related fitness factors of a measurer stored in the health-related fitness measurement information storage (310); and a measurement method guide (330) that enables a measurer to easily understand the health-related fitness factors to be measured, by outputting description and measurement methods of the health-related fitness factors to be measured through the health-related fitness information display (320) using any one or more of sound, a picture, and a moving image.

The health-related fitness measurement information storage (310) includes: a body configuration measurement information storage module (311) that stores measurement information and result of a measurer transmitted from the body configuration measurement data transmitter (212); a flexibility measurement information storage module (312) that stores measurement information and result of a measurer transmitted from the flexibility measurement data transmitter (222); a cardiopulmonary endurance measurement information storage module (313) that stores measurement information and result of a measurer transmitted from the cardiopulmonary endurance measurement data transmitter (232); a muscular strength measurement information storage module (314) that stores measurement information and result of a measurer transmitted from the muscular strength measurement data transmitter (242); and a muscular endurance measurement information storage module (315) that stores measurement information and result of a measurer transmitted from the muscular endurance measurement data transmitter (252).

Accordingly, the measurement information and result for each of the health-related fitness factors of the measurer measured by the health-related fitness measurement device (200) are stored.

The measurement method guide (330) includes: a body configuration measurement method guide module (331) that stores information about the measurement method for measuring a body configuration, and if necessary, outputs the information to a measurer; a flexibility measurement method guide module (332) that stores information about the measurement method for measuring flexibility, and if necessary, outputs the information to a measurer; a cardiopulmonary endurance measurement method guide module (333) that stores information about the measurement method for measuring cardiopulmonary endurance, and if necessary, outputs the information to a measurer; a muscular strength measurement method guide module (334) that stores information about the measurement method for measuring muscular strength, and if necessary, outputs the information to a measurer; and a muscular endurance measurement method guide module (335) that stores information about the measurement method for measuring muscular endurance, and if necessary, outputs the information to a measurer.

Accordingly, the measurer is able to easily understand the measurement methods, whereby accurate measurement results are derived from correct measurement postures.

On the other hand, the integrated measurement system (1) for measuring health-related fitness factors further includes a measurement result communication unit (N) that shares and transmits the measurement date, personal information, and the measurement result of a measurer to a database (DB1) of a measurement organization, a database (DB2) of a public institution, a dedicated terminal (S1) of a manager, and a personal terminal (S2) of the measurer.

Accordingly, it is possible to induce the measurement process and result of the health-related fitness factors of the measurer to be professionally managed, and it is also possible to construct indexes for healthcare on the basis of the collected measurement data.

The measurement result communication unit (N) includes: a wire communication unit (N1) that connects the dedicated terminal (S1) of the manager and the personal terminal (S2) of the measurer to the measurement information outputter (300) through a cable, if necessary, so that information can be transferred among them; and a wireless communication unit (N2) that wirelessly connects the database (DB1) of the measurement organization, the database (DB2) of the public institution, the dedicated terminal (S1) of the manager, and the personal terminal (S2) of the measurer to the measurement information outputter (300), if necessary, so that information can be transferred among them.

Further, the system further includes a measurement order provider (B) that, when the health-related fitness factors are measured through the health-related fitness measurement device (200), turns on/off the health-related fitness measurement device (200) to be used for measurement as the measurement method guide (330) is output in accordance with the measurement order so that a measurer can more easily use the health-related fitness measurement device (200).

Accordingly, a measurer can more easily perform measurement.

That is, it is possible to further induce the effect of the measurement method guide (330).

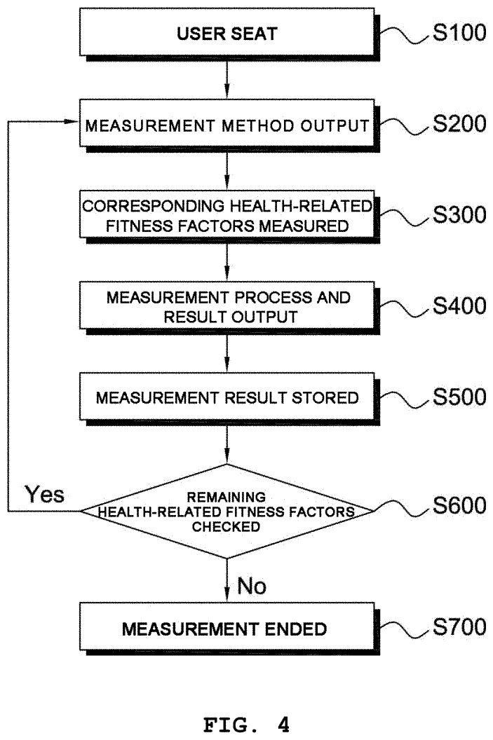

On the other hand, FIG. 4 is a flowchart simply showing the procedure of measuring health-related fitness factors using the integrated measurement system (1) for measuring health-related fitness factors of the present invention.

As shown in FIG. 4, a measurer sits in the user seat (100) to measure various health-related fitness factors ( [symbol] S100, measurer-sitting step),

when it is determined that the measurer has sit, a guide for the health-related fitness factors to be measured is output through the measurement information outputter (300) ( [symbol] S200, measurement method-outputting step), and

the measurer is enabled to measure the health-related fitness factors through the health-related fitness measurement device (200) in accordance with the output guide ( [symbol] S300, health-related fitness factor-measuring step).

In this procedure, the measurement process and result of the health-related fitness factors that are measured from the measurer are output in real time to the measurement information outputter (300) ( [symbol] S400, measurement process and result-outputting step), and

the measured result is stored ( [symbol] S500, measurement result-storing step),

so that other health-related fitness factors to be sequentially measured are induced to be measured ( [symbol] S600, remaining health-related fitness factor-checking step).

Measurement is ended when there is no more health-related fitness factor to be measured ( [symbol] S700, measurement-ending step),

whereby measuring various health-related fitness factors of the measurer is finished.

On the other hand, the measurement controller (400) that controls the user seat (100), the health-related fitness measurement device (200), and the measurement information outputter (300) enables the mechanical operation of the user seat (100) and the health-related fitness measurement device (200), and corresponding measurement process, result, and output by the health-related fitness measurement device (200) and the measurement information outputter (300) to be stably performed.

Consequently, measurement information obtained from the integrated measurement system (1) for measuring health-related fitness factors of the present invention can be collected into one program through wire and wireless communication networks, and the collected personal measurement data can be reflected to periodic evaluation and can be used as personal healthcare indexes through periodic evaluation data. Further, indexes that are lowered or abnormal in the healthcare indexes are sent to a special management organization or special medical institution to be able to receive follow-up management. Further, the information can be used later as healthcare indexes for the nation by sharing the measured information with National Health Insurance Corporation.

Further, the measurement information outputter (300) of the components of the integrated measurement system (1) for measuring health-related fitness factors of the present invention further includes an anti-fraud unit (TD) for preventing a fraud by a measurer or a fraud by a unauthenticated person.

Accordingly, it is possible to derive objective measurement process and result for health-related fitness factors.

That is, when a measurer sits in the user seat (100) to measure health-related fitness factors, the integrated measurement system (1) for measuring health-related fitness factors of the present invention can measure five health-related fitness factors with one-time sitting due to the characteristics of the system. Accordingly, it is possible to prevent a fraud by a measurer and an unauthenticated user using the characteristics of the system.

Describing this configuration in more detail in terms of the components, the system includes: a measurer authentication module (TD1) that checks, examines, and stores identification information (TM) of a target sitting in the user seat (100) for measurement; a measurer measurement process-temporal storage module (TD2) that temporarily stores measurement process and result of health-related fitness factors of the measurer authenticated by the measurer authentication module (TD1); a measurement-temporal stop module (TD3) that delays measurement for a predetermined time when a measurer leaves the user seat (100) for an avoidable reason while measuring health-related fitness factors; a measurement-continuing module (TD4) that deactivates the measurement-temporal stop module (TD3) and enables measurement to continue; and a measurer measurement result connection module (TD5) that connects the measurement process and result of five health-related fitness factors of the measurer to the health-related fitness measurement information storage (310).

Accordingly, the objectivity of the measurement process and result of the health-related fitness factors of the measurer is maximized.

In this configuration, as described above, when a measurer has to unavoidably leave the user seat (100) while measuring the health-related fitness factors, the measurement-temporal stop module (TD3) is activated, and when the measurer sits again in the user seat (100) for measurement, the identification information (TM) of the measurer is checked and examined by the measurer authentication module (TD1) and measurement is restarted and continued.

Accordingly, it is possible to obtain measurement process and result with maximized objectivity by preventing a fraud of a measurer and an unauthenticated user when measuring health-related fitness factors.

For reference, the term ‘health-related fitness’ used herein means muscular strength, muscular endurance, flexibility, cardiopulmonary endurance, and a body configuration (body composition examination).

Further, the term ‘identification information (TM)’ means information enabling identification and recognition of a measurer such as the fingerprints, face, iris, weight, resident registration number of a measurer.

In the configuration and operations, according to the present invention described above, it can easily measure various health-related fitness factors as one measurement system at the same position without specifically moving a measurer and a measurement device.

Further, the present invention enables a measurer to monitor in real time measurement process and result of his/her health-related fitness factors while measuring the health-related fitness factors.

Further, the present invention can provide a healthful mental stimulus so that a measurer can be given and maintain a motive about health management as the system enables the measurer to monitor his/her health-related fitness status in real time when measuring the health-related fitness factors.

Further, the present invention can be easily managed and measured by a manager and a measurer by securing convenience in management and measurement of a measurement device and objectivity in measurement process and result.

Further, the present invention can be considered as a very effective invention that can specially manage the measurement date, personal information, and measurement data of a measurer by sharing and transmitting them to a database of a measurement organization, a database of a public institution, a dedicated terminal of a manager, and a personal terminal of the measurer, can later construct indexes for healthcare using measurement data for health-related fitness factors collected, and can improve the health of the nation (induce a measurer to steadily exercise by making the measurer feel the necessity of healthcare on the basis of a measurement data).

Further, since various health-related fitness factors are measured and examined in one integrated measurement system, frauds such as changing the result or measuring the factors by another person rather than the subjected person, and an unauthenticated user are prevented, thereby outputting and storing an objective and clear measurement result of health-related fitness factors of a measurer.

It will be apparent to those skilled in the art that various modifications and variations can be made in the present invention without departing from the spirit or scope of the invention as defined in the appended claims.

This invention can be implemented in many different forms without departing from technical aspects or main features. Therefore, the implementation examples of this invention are nothing more than simple examples in all respects and will not be interpreted restrictively.

### INDUSTRIAL APPLICABILITY

The present invention relates to an integrated measurement system for measuring health-related fitness factor and can be applied to contribute to the promotion of a variety of medical industry and fitness industry such as manufacturing and sales industries thereof, a health examination center for checking of the status of the health-related fitness by using it, a training center, and a rehabilitation sports center etc.

What is claimed is:

1. An integrated measurement system (1) for measuring health-related fitness factors, the integrated measurement system comprising:
a user seat (100) enabling a measurer to sit and measure health-related fitness factors;
a health-related fitness measurement device (200) measuring health-related fitness factors of the measurer sitting in the user seat;
a measurement information outputter (300) outputting in real time measurement process and result of health-related fitness factors of the measurer that are measured by the health-related fitness measurement device (200) so that the measurer and a manager can monitor; and
a measurement controller (400) controlling the user seat (100), the health-related fitness measurement device (200), and the measurement information outputter (300),
wherein the health-related fitness measurement device (200) includes:
a body configuration measurement unit (210) that quantitatively measure body composition for each of a muscle amount and a body fat amount of the measurer;
a flexibility measurement unit (220) that measures the degrees of maintenance and increase of the available ranges of joints and muscles of the measurer;
a cardiopulmonary endurance measurement unit (230) that measures the ability to maintain the function of the circulatory system of the heart and the respiratory system of the lung of the measurer;
a muscular strength measurement unit (240) that measures the strength of the muscles of the measurer and durability of the strength; and
a muscular endurance measurement unit (250) that measures durability of predetermined muscular strength of a measurer for a long period of time, and
several health-related fitness factors of the measurer are easily measured through one system without moving the measurer and replacing measurement devices.

2. The integrated measurement system (1) of claim 1, further comprising a measurement result communication unit (N) that shares and transmits a measurement date, personal information, and a measurement result of the measurer to a database (DB1) of a measurement organization, a database (DB2) of a public institution, a dedicated terminal (S1) of a manager, and a personal terminal (S2) of the measurer,
whereby the measurement process and result of the health-related fitness factors of the measurer are induced to be professionally managed and indexes for healthcare are constructed on the basis of the collected measurement data.
